(19) United States
(12) Patent Application Publication
(10) Pub. No.: US 2024/0071631 A1
(43) Pub. Date: Feb. 29, 2024

(54) ARTIFICIAL INTELLIGENCE-AIDED FAMILY HEALTH CARE SYSTEM

(71) Applicants: Laphoi Philip Chu, Beijing (CN); Wei Cao, Beijing (CN)

(72) Inventors: Laphoi Philip Chu, Beijing (CN); Wei Cao, Beijing (CN)

(21) Appl. No.: 17/506,655

(22) Filed: Oct. 20, 2021

(51) Int. Cl. G16H 80/00; G16H 50/30

(52) U.S. Cl. CPC G16H 80/00; G16H 50/30

(57) ABSTRACT

An AI-aided family health care system is disclosed. The health care system includes an AI cloud engine subsystem which is connected with a user mobile application program through an first interface. The AI cloud engine subsystem is connected with a third-party platform through an second interface. The AI cloud engine subsystem is connected with an operation management interface through a https. The AI cloud engine subsystem is connected with a home gateway through an third interface. The AI cloud engine subsystem is connected with a health monitoring subsystem and an environment monitoring subsystem through the home gateway.

### TECHNICAL FIELD

The present disclosure relates to the technical field of health care, and particularly relates to an artificial intelligence (AI)-aided family health care system.

### BACKGROUND

Modern families tend to be smaller, and the members of the extended family, such as parents, brothers and sisters usually do not live together. There is lack of interaction between family members in terms of health and life styles, leading to a low happiness index of family life and even slow response from the family members in case of accidents in life.

Through search, the Chinese patent with patent No. CN109903825A discloses a health management system based on artificial intelligence, including a data acquisition module configured to record user information; a data cleaning module, a clustering module, a health management prediction module and an importance assessment module of health management factors.

The above patent has the following defects: devices and systems are designed for individual device users, and the understanding and feedback of user family members on the health status of the device users are not incorporated into the analysis and interpretation of health index data. Therefore, an AI-aided family health care system is proposed.

### SUMMARY

The purpose of the present disclosure is to provide an AI-aided family health care system in order to solve the defects in the prior art, which incorporates the understanding and feedback of user family members on the health status of device users into the analysis and interpretation of health index data to form a more comprehensive and accurate family health care service.

To achieve the above purpose, the present disclosure adopts the following technical solution:

An AI-aided family health care system includes an AI cloud engine subsystem which is connected with a user mobile application program through first interface. The AI cloud engine subsystem is connected with a third-party platform through second interface. The AI cloud engine subsystem is connected with an operation management interface through https. The AI cloud engine subsystem is connected with a home gateway through a third interface. And the AI cloud engine subsystem is connected with a health monitoring subsystem and an environment monitoring subsystem through the home gateway.

Preferably, the AI cloud engine subsystem includes a health index interaction combination customized by family members and interaction indexes of other family members; the health index interaction combination comprises heart rate, respiration, blood oxygen, blood pressure, body temperature, temperature, humidity, smoke, brightness and exercises, sleep time, water, electricity and gas switches, door opening and closing status induction, window opening and closing status induction, light switch, stove switch status induction, refrigerator door opening and closing status induction, water leakage induction, gas leakage induction and air quality induction; and the interaction indexes of other family members comprise attention indexes, attention time and attention frequency.

Preferably, the health monitoring subsystem includes a exercises sensor, a pulse and blood pressure measuring module, a blood oxygen and blood glucose measuring module, a body temperature measuring module, an emergency button, and an emergency call and fall sensing module.

Preferably, the environment monitoring subsystem includes a temperature sensor, a smoke sensor, an intelligent illuminating lamp, an intelligent switch, water, electricity and gas switches, door opening and closing status induction, window opening and closing status induction, a light switch, stove opening and closing status induction, refrigerator door opening and closing status induction, water leakage induction, gas leakage induction and air quality induction.

Preferably, the home gateway is configured to conducts personalized configuration for various devices and modules of the health monitoring subsystem and the environment monitoring subsystem, and transmits the statuses and data of the devices and the modules back to the AI cloud engine subsystem for analysis, statistics and interpretation.

Preferably, the AI cloud engine subsystem forms an interactive closed loop with the mobile application program on mobile phones of family members of a user.

Preferably, the home gateway, the user mobile application program and the emergency call module conduct voice communication via Bluetooth, a wireless network or a mobile communication network through scheduling of the AI cloud engine subsystem.

Preferably, a use method of the AI-aided family health care system includes the following steps:

S1: downloading the user mobile application program with a user mobile phone and the mobile phones of the user family members, and connecting the user mobile phone with the AI cloud engine subsystem through the wireless network or the mobile communication network;

S2: conducting personalized configuration by the user on various devices and modules of the health monitoring subsystem and the environment monitoring subsystem through the home gateway;

S3: reporting, by the user mobile phone, the sensor data in a Bluetooth device to cloud health data through a Bluetooth gateway and the wireless network or mobile communication, receiving a cloud control command, and receiving a management command of the user mobile application program through a cloud; and

S4: connecting the user mobile application program in the mobile phone of the user family members with the AI cloud engine subsystem through the mobile communication network.

Preferably, operatable items of the user family members in the mobile application program includes a customized overview panel, a customized home page, personalized customization parameters, attention points, an operation path, attention time and attention frequency.

The present disclosure has the following beneficial effects:

1. The present disclosure introduces the interaction of family members into the artificial intelligence analysis of health data to form a closed loop, which not only can improve the accuracy of interpreting the health statuses of the elderly and the disabled in a family, but also promotes benign interaction among the family members in terms of health and safety, and improves the overall happiness of family life.

2. The present disclosure introduces the understanding of other family members for the health condition of the concerned family members into the analysis and interpretation of the health data, enhances accuracy and pertinence of analysis results, reminds the family member users through customization to interact with each other to create a good family care environment and improves the happiness and health level of family life.

### DESCRIPTION OF DRAWINGS

FIG. 1 is a structural schematic diagram of the AI-aided family health care system in the present disclosure; and

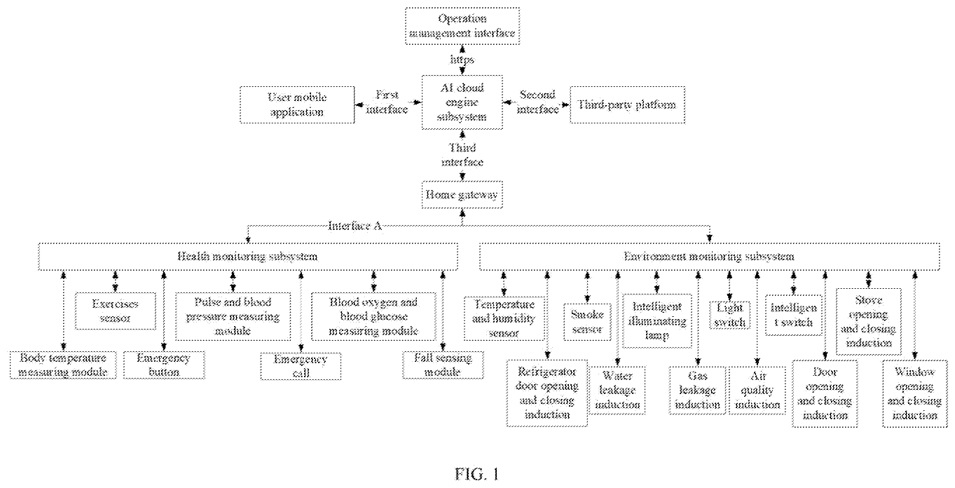

FIG. 2 is a structural schematic diagram of a use method of the AI-aided family health care system in the present disclosure.

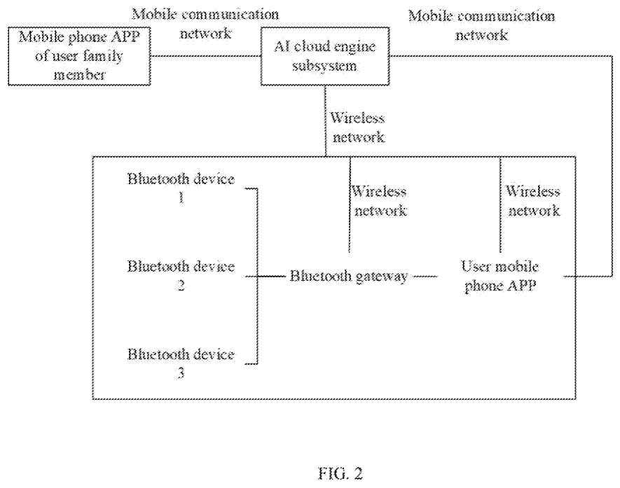

### DETAILED DESCRIPTION

A technical solution of the disclosure is further described in detail below in combination with specific embodiments.

### Embodiment 1

An AI-aided family health care system includes an AI cloud engine subsystem which is connected with a user mobile application program through a first interface. The AI cloud engine subsystem is connected with a third-party platform through second interface. The AI cloud engine subsystem is connected with an operation management interface through https. The AI cloud engine subsystem is connected with a home gateway through a third interface. And the AI cloud engine subsystem is connected with a health monitoring subsystem and an environment monitoring subsystem through the home gateway.

The AI cloud engine subsystem includes a health index interaction combination customized by family members and interaction indexes of other family members. The health index interaction combination includes heart rate, respiration, blood oxygen, blood pressure, body temperature, temperature, humidity, smoke, brightness and exercises, sleep time, and water, electricity and gas switches; and the interaction indexes of other family members comprise attention indexes, attention time and attention frequency.

A exercises sensor, a pulse and blood pressure measuring module, a blood oxygen and blood glucose measuring module, a body temperature measuring module, an emergency button, and an emergency call and fall sensing module are arranged in the health monitoring subsystem. The health monitoring subsystem is used to detect distress call information initiated by a designated family member actively and receive the health data of the family member obtained through a Bluetooth bracelet, such as exercise, sleep, heart rate, respiration, blood oxygen, blood pressure and body temperature.

The environment monitoring subsystem is provided with a temperature sensor, a smoke sensor, an intelligent illuminating lamp and an intelligent switch. The environment monitoring subsystem is used to detect the living environment of the designated family member, such as temperature, humidity, smoke, brightness and water, electricity and gas switch conditions.

The home gateway conducts customized configuration for various devices and modules of the health monitoring subsystem and the environment monitoring subsystem, and transmits the statuses and data of the devices and the modules back to the AI cloud engine subsystem for analysis, statistics and interpretation.

The AI cloud engine subsystem forms an interactive closed loop with the mobile application program on mobile phones of family members of a user.

The home gateway, the user mobile application program and the emergency call module conduct voice communication via Bluetooth, a wireless network or a mobile communication network through scheduling of the AI cloud engine subsystem.

### Embodiment 2

An AI-aided family health care system is provided, with a use method including the following steps:

S1: downloading the user mobile application program with a user mobile phone and the mobile phones of the user family members, and connecting the user mobile phone with the AI cloud engine subsystem through the wireless network or the mobile communication network;

connecting the user mobile application program in the user mobile phone with the AI cloud engine subsystem through the wireless network or the mobile communication network to acquire cloud health data, and controlling a Bluetooth network through cloud management;

S2: conducting personalized configuration by the user on various devices and modules of the health monitoring subsystem and the environment monitoring subsystem through the home gateway;

S3: reporting, by the user mobile phone, the sensor data in a Bluetooth device to cloud health data through a Bluetooth gateway and the wireless network or mobile communication, receiving a cloud control command, and receiving a management command of the user mobile application program through a cloud; and

S4: connecting the user mobile application program in the mobile phone of the user family member with the AI cloud engine subsystem through the mobile communication network;

receiving health data of relatives and friends, by the user mobile application program in the mobile phones of family members of a user.

The user mobile application program introduces the customization setting of the family members on the interface of the user mobile application program into the AI cloud engine subsystem. The data from the user mobile application program is combined with the data transmitted back by the health monitoring subsystem and the environment monitoring subsystem through the home gateway to form a personalized dynamic database that is updated in real time. The home gateway, the user mobile application program and the emergency call module conduct voice communication via Bluetooth, the wireless network or the mobile communication network through scheduling of the AI cloud engine subsystem. And the AI cloud engine subsystem forms a closed loop for the health data in the artificial intelligence analysis, and reminds the family member users to interact with each other through customization.

The above are just preferred embodiments of the present disclosure, but the protection scope of the present disclosure is not limited thereto. Any equivalent replacement or change made by those skilled in the art familiar with the technical field within the technical scope disclosed by the present disclosure shall be covered within the protection scope of the present disclosure.

1. An artificial intelligence (AI)-aided family health care system, comprising an AI cloud engine subsystem connected with a user mobile application program through a first interface; wherein the AI cloud engine subsystem is connected with a third-party platform through a second interface; the AI cloud engine subsystem is connected with an operation management interface through a https; the AI cloud engine subsystem is connected with a home gateway through a third interface; and the AI cloud engine subsystem is connected with a health monitoring subsystem and an environment monitoring subsystem through a home gateway.

2. The AI-aided family health care system of claim 1, wherein the AI cloud engine subsystem comprises a health index interaction combination customized by family members and interaction indexes of other family members; the health index interaction combination comprises heart rate, respiration, blood oxygen, blood pressure, body temperature, temperature, humidity, smoke, brightness and exercises, sleep time, water, electricity and gas switches, door opening and closing status induction, window opening and closing status induction, light switch, stove switch status induction, refrigerator door opening and closing status induction, water leakage induction, gas leakage induction and air quality induction; and the interaction indexes of other family members comprise attention indexes, attention time and attention frequency.

3. The AI-aided family health care system of claim 1, wherein the health monitoring subsystem comprises an exercises sensor, a pulse and blood pressure measuring module, a blood oxygen and blood glucose measuring module, a body temperature measuring module, an emergency button, and an emergency call and fall sensing module.

4. The AI-aided family health care system of claim 1, wherein the environment monitoring subsystem comprises a temperature sensor, a smoke sensor, an intelligent illuminating lamp, an intelligent switch, water, electricity and gas switches, door opening and closing status induction, window opening and closing status induction, a light switch, stove opening and closing status induction, refrigerator door opening and closing status induction, water leakage induction, gas leakage induction and air quality induction.

5. The AI-aided family health care system of claim 1, wherein the home gateway is configured to conduct personalized configuration for various devices and modules of the health monitoring subsystem and the environment monitoring subsystem, and transmits the statuses and data of the devices and the modules back to the AI cloud engine sub system for analysis, statistics and interpretation.

6. The AI-aided family health care system of claim 1, wherein the AI cloud engine subsystem forms an interactive closed loop with the mobile application program on mobile phones of family members of a user.

7. The AI-aided family health care system of claim 1, wherein the home gateway, the user mobile application program and the emergency call module conduct voice communication via Bluetooth, a wireless network or a mobile communication network through scheduling of the AI cloud engine subsystem.

8. An artificial intelligence-aided family health care system, with a use method comprising the following steps:
S1: downloading the user mobile application program with a user mobile phone and the mobile phones of the user family members, and connecting the user mobile phone with the AI cloud engine subsystem through the wireless network or the mobile communication network;
S2: conducting customized configuration by the user on various devices and modules of the health monitoring subsystem and the environment monitoring subsystem through the home gateway;
S3: reporting, by the user mobile phone, the sensor data in a Bluetooth device to cloud health data through a Bluetooth gateway and the wireless network or mobile communication, receiving a cloud control command, and receiving a management command of the user mobile application program through a cloud; and
S4: connecting the user mobile application program in the mobile phones of the user family members with the AI cloud engine subsystem through the mobile communication network.

9. The method of claim 8, the mobile application program of the artificial intelligence-aided family health care system, with an optional customized use method comprising the following steps:
S1: configure a customized overview panel.
S2: configure a customized home page.
S3: configure a set of customized combination of parameters, concerns, operation path, attention schedule, attention span and attention frequency.
